(19) United States
(12) Patent Application Publication
(10) Pub. No.: US 2019/0303488 A1
(43) Pub. Date: Oct. 3, 2019

(54) MULTI CHARACTER SET CONVERSION

(71) Applicant: Konica Minolta Healthcare Americas, Inc., Wayne, NJ (US)

(72) Inventor: Hiroyuki Kubota, Wayne, NJ (US)

(73) Assignee: Konica Minolta Healthcare Americas, Inc., Wayne, NJ (US)

(21) Appl. No.: 15/942,216

(22) Filed: Mar. 30, 2018

(51) Int. Cl. G06F 17/30; G16H 30/20

(52) U.S. Cl. CPC G06F 17/30569; G16H 30/20; G06F 17/30864; G06F 17/3028

(57) ABSTRACT

A management server that communicates with a plurality of medical devices includes a memory that stores a plurality of medical records each comprising characters encoded in a first encoding; and a processor that: receives a request from one of the plurality of medical devices; detects a second encoding used to encode characters included in the request; converts the characters included in the request to the first encoding; searches the plurality of medical records with the converted characters; converts a search result of the plurality of medical records to the second encoding; and transmits the search result to the medical device.

### BACKGROUND

Electronic medical records, including medical images and other medical data play a crucial role in the diagnosis of patients. Healthcare facilities (e.g., hospitals) have realized the benefits of electronically storing medical records. The digitalization of medical images and other data not only enables users to easily access the medical images and medical data, but also enables the images and data to be easily shared between multiple healthcare facilities.

In the healthcare industry, the use of a system known as a Picture Archiving and Communications System (“PACS”) is becoming increasingly popular for convenient storage and access of medical images and reports. Generally, a PACS comprises a multitude of devices working cooperatively to digitally capture, store, manage, distribute, and display medical images generated by various imaging modalities, such as computed tomography (CT), magnetic resonance imaging (MRI), position emission tomography (PET), ultrasound, X-ray, etc. PACS allows various healthcare facilities to share all types of images and reports captured or created internally or externally.

In general, it is sufficient for PACS to only support the language used in the country where the medical facility is located. However, because of the recent trend of globalization, there is a situation where two or more languages are used in a single medical facility or a group that operates several medical facilities in a certain area. For example, a medical group in the U.S. has started to use both English and Spanish for managing patients' medical records in its medical system.

### SUMMARY

In general, the invention relates to a medical imaging system.

In one aspect according to one or more embodiments of the invention, a management server that communicates with a plurality of medical devices includes a memory that stores a plurality of medical records each comprising characters encoded in a first encoding; and a processor that: receives a request from one of the plurality of medical devices; detects a second encoding used to encode characters included in the request; converts the characters included in the request to the first encoding; searches the plurality of medical records with the converted characters; converts a search result of the plurality of medical records to the second encoding; and transmits the search result to the medical device (i.e., the one of the plurality of medical devices).

In another aspect according to one or more embodiments of the invention, a method for a management server that communicates with a plurality of medical devices includes storing a plurality of medical records each comprising characters encoded in a first encoding; receiving a request from one of the plurality of medical devices; detecting a second encoding used to encode characters included in the request; converting the characters included in the request to the first encoding; searching the plurality of medical records with the converted characters; converting a search result of the plurality of medical records to the second encoding; and transmitting the search result to the medical device (i.e., the one of the plurality of medical devices).

In another aspect according to one or more embodiments of the invention, a non-transitory computer readable medium (CRM) storing instructions for a management server that communicates with a plurality of medical devices, the instructions include functionality for: storing a plurality of medical records each comprising characters encoded in a first encoding; receiving a request from one of the plurality of medical devices; detecting a second encoding used to encode characters included in the request; converting the characters included in the request to the first encoding; searching the plurality of medical records with the converted characters; converting a search result of the plurality of medical records to the second encoding; and transmitting the search result to the one of the plurality of medical device.

Other aspects of the invention will be apparent from the following description and the appended claims.

### BRIEF DESCRIPTION OF DRAWINGS

FIG. 1 shows a medical system in accordance with one or more embodiments of the invention.

FIG. 2 shows a block diagram of a medical system in accordance with one or more embodiments of the invention.

FIGS. 3-5 each show a flowchart in accordance with one or more embodiments of the invention.

FIGS. 6-9 each show an example of a screen displayed by a medical system in accordance with one or more embodiments of the invention.

FIG. 10 shows a computer system in accordance with one or more embodiments of the invention.

### DETAILED DESCRIPTION

Specific embodiments of the invention will now be described in detail with reference to the accompanying figures. Like elements in the various figures are denoted by like reference numerals for consistency.

In the following detailed description of embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers does not imply or create a particular ordering of the elements or limit any element to being only a single element unless expressly disclosed, such as by the use of the terms “before,” “after,” “single,” and other such terminology. Rather, the use of ordinal numbers is to distinguish between the elements. By way of an example, a first element is distinct from a second element, and the first element may encompass more than one element and succeed (or precede) the second element in an ordering of elements.

It is to be understood that one or more of the steps shown in the flowcharts may be omitted, repeated, and/or performed in a different order than the order shown. Accordingly, the scope of the invention should not be considered limited to the specific arrangement of steps shown in the flowcharts.

In the following detailed description of embodiments of the invention, numerous specific details are set forth to provide a more thorough understanding of the invention. However, it will be apparent to one of ordinary skill in the art that the invention may be practiced without these specific details. In other instances, well-known features have not been described in detail to avoid unnecessarily complicating the description.

In general, one or more embodiments of the invention provide a management server, a method, and a non-transitory computer readable medium for storing and managing electronic medical records in a medical system such as a PACS where two or more languages are used to input the records to the system. The management server of the medical system in accordance with one or more embodiments of the invention enables various kinds of medical devices, such as medical imaging devices, medical user terminals, and other management servers to be integrated into a single system even when these medical devices each use a different language or encoding. Moreover, the management server allows a medical person to use the default language in his user terminal to request and retrieve medical information, such as medical images and medical report without considering the difference of the language or encoding used in the medical system.

In one or more embodiments of the invention, the management server stores medical records in Unicode, encoded by a predetermined encoding such as UTF-8. When receiving a request for the medical records from one of the medical devices (e.g., a user terminal), the management server detects the encoding of the characters included in the request such as a patient name, and responds to the request by: converting the encoding of the received characters to the predetermined encoding (i.e., UTF-8), searching the medical records with the converted characters, and converting the encoding of the search result back to the encoding used in the request. According to this configuration, the medical device can transmit a request for medical content search without considering the difference of the encoding, which advantageously helps the medical device that operates in a different language communicate with the management server without any software update or modification.

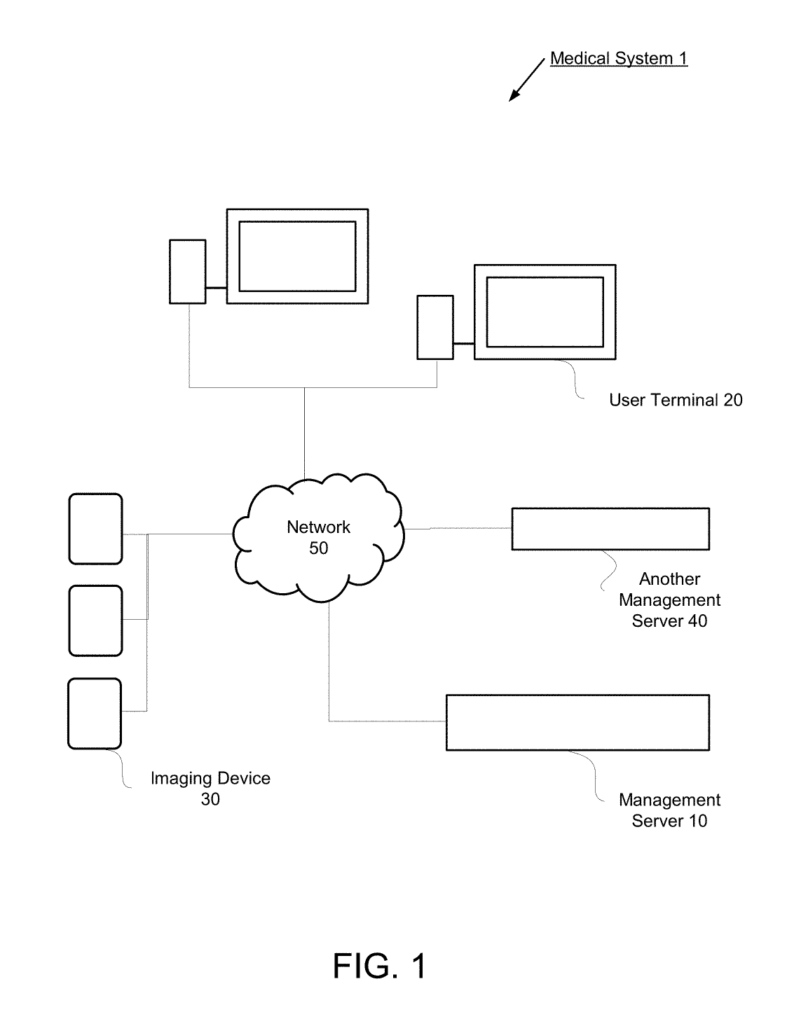

FIG. 1 shows a medical system 1 in accordance with one or more embodiments of the invention. The medical system 1 includes a management server (hereinafter “first management server”) 10, a user terminal 20, an imaging device 30, and another management server (hereinafter “second management server”) 40. These medical devices are connected via a network 50 and installed in a medical facility or over a plurality of medical facilities.

In one or more embodiments of the invention, the medical system is a PACS. The first management server 10 and the other medical devices 20, 30, and 40 exchange medical data with a predetermined medical data format, such as Digital Imaging and Communications in Medicine (DICOM) and Health Level Seven International (HL7).

For example, a DICOM file contains a request (or command) to retrieve a list of medical records stored in the first management server 10 and search criteria for filtering the medical records to be searched. Such search criteria include, but are not limited to, a patient ID, a patient name, and date of birth. The keywords specified as the search criteria are encoded in a default encoding of each of the medical devices. The first management server 10 detects the character encoding used in the DICOM file (e.g., GB18030 for Chinese) and converts the encoding to a predetermined encoding (e.g., UTF-8) being used in the medical database. The first management server 10 then executes the database search using the converted characters, and transmits the result after converting the encoding of the result back to the original encoding (i.e., GB18030).

In one or more embodiments of the invention, the first management server 10 may receive from the other medical devices a request for transmitting a medical image, a medical image order, a medical image interpretation report, or a medical examination report, as specified by search criteria. Similar to the case of the request for retrieving the content list, the first management server 10 may detect the encoding of the keywords used to specify the medical image, order, or report, and then transmit the search result in the encoding used in the request. As stated above, the internal search of the medical records in the first management server 10 is executed in a predetermined encoding (e.g., UTF-8), and the result is transmitted with the original encoding used in the request.

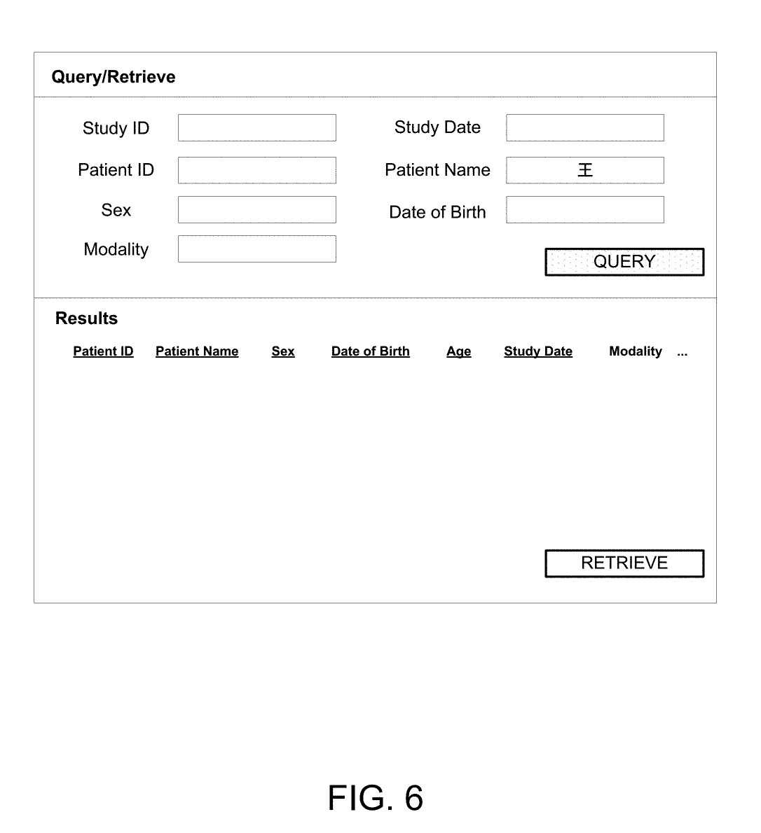

The user terminal 20 in accordance with one or more embodiments of the invention is a medical user terminal operated by a medical person or user (hereinafter called “user”). The user terminal 20 has a display and an input device so that the user can access the medical records stored in the first management server 10 by specifying characters as search criteria. FIG. 6 shows an example of a screen shown on the display of the user terminal 20, which allows the user to specify the criteria, such as a patient ID, a patient name, and date of birth. When one or more of these criteria are set and the “QUERY” button is clicked, the user terminal 20 transmits to the first management server 10 a request for retrieving a list of medical records stored in the first management server 10, that meet the search criteria, in the form of a DICOM file.

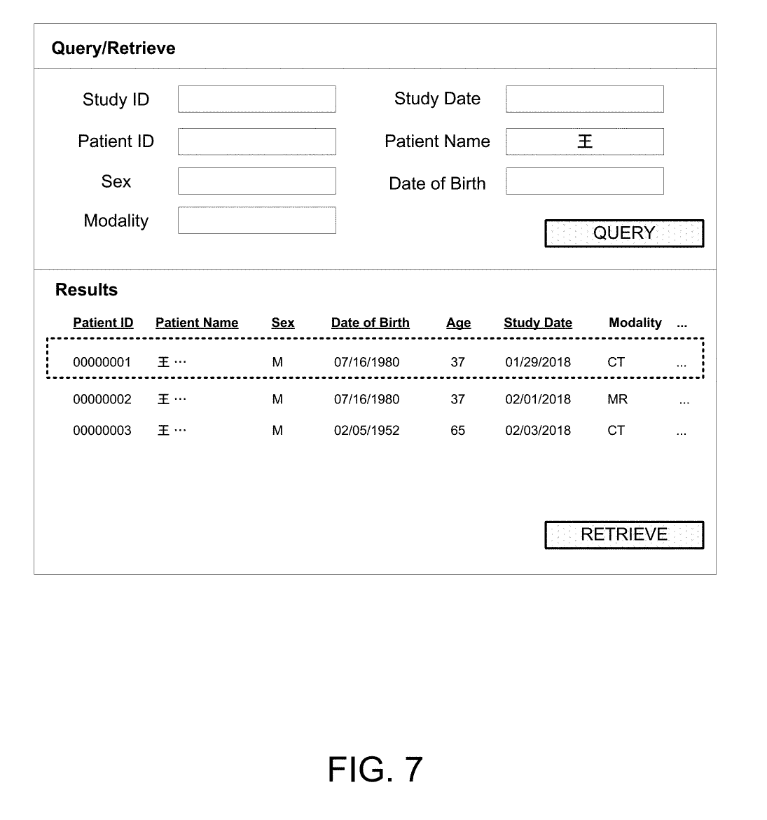

Additionally, in response to the criteria specified by the user, the user terminal 20 may transmit to the first management server 10 a request for retrieving a medical record including a medical image, a medical image interpretation report, or a medical examination report. The user terminal 20 may transmit the request in the form of the DICOM file. FIG. 7 shows an example of a screen shown on the display of the user terminal 20, which allows the user to specify a desired medical record. When the record is selected and the “RETRIEVE” button is clicked, the user terminal 20 transmits to the first management server 10 a request for retrieving the specified medical record.

The imaging device 30 in accordance with one or more embodiments of the invention is an imaging modality, such as Computed Radiography (CR), CT, and MRI. In addition to such imaging function, the imaging device 30 is capable of accepting an input from the user and communicating with the first management server 10 for retrieving a medical record including a medical image and a medical image order in the form of a DICOM file.

The other (second) management server 40 in accordance with one or more embodiments of the invention may transmit to the first management server 10 a request for retrieving a list of medical records stored in the first management server 10 or a medical image, a medical image order, a medical image interpretation report, and a medical examination report, in the form of the DICOM file. The request from the other management server 40 may contain characters as search criteria encoded in an encoding different from the one used in the first management server 10.

In one or more embodiments of the invention, the network 50 is formed by one or more networks including an intranet deployed in a medical facility and/or the Internet.

According to this configuration, the first management server 10 allows various kinds of existing medical devices that handle characters in different encodings to be integrated into a system without modifying existing software or implementing any additional software for each of the medical devices.

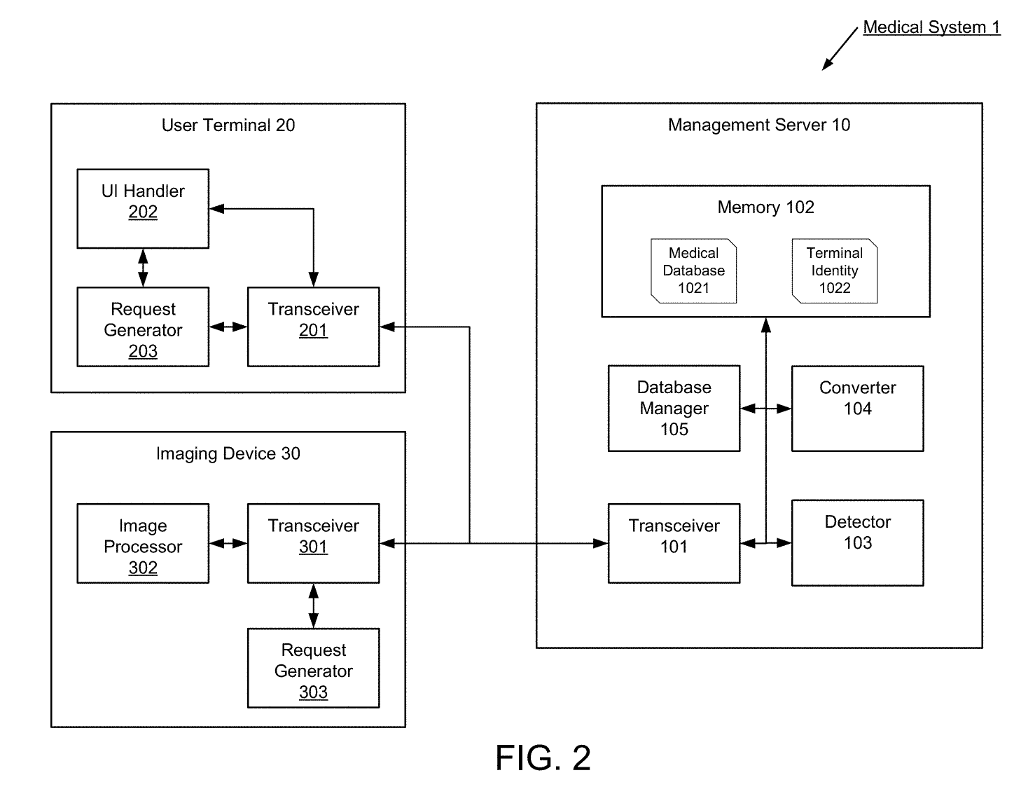

FIG. 2 shows a block diagram of a medical system in accordance with one or more embodiments of the invention.

As shown in FIG. 2, the first management server 10 of the medical system 1 includes a transceiver 101, a memory 102, a detector 103, a converter 104, and a database manager 105.

The transceiver 101 of the first management server 10 communicates with another medical device such as the user terminal 20 and the imaging device 30. The transceiver 101 receives from such a medical device in the form of a DICOM file, and transmits a response to the medical device. The response includes, but not limited to, a simple acknowledgement that indicates the success of the communication and a response including a search result based on a specific request for retrieving medical records including medical images, medical image orders, medical image interpretation reports, and medical examination reports. The response may be transmitted in the form of the DICOM file like the request from the other medical device.

The memory 102 of the first management server 10 stores medical database 1021 for managing medical records and a terminal identity 1022 that identifies a medical device communicating with the first management server 10. In one or more embodiments of the invention, the medical records include medical images associated with their attributes (e.g., a patient ID, a patient name, a date of birth, a study description, an image comment, etc.), medical image orders, medical image interpretation reports, and medical examination reports. In one or more embodiments of the invention, the characters contained in these medical records are encoded in a predetermined common encoding such as UTF-8.

When the transceiver 101 receives a request from a medical device in the form of the DICOM file, the detector 103 of the first management server 10 parses the DICOM file and detects the encoding of the characters included in the DICOM file. In one or more embodiments of the invention, the detector 103 parses characters surrounded by tags of the DICOM file to find predetermined characters indicating the encoding of the included characters, such as GB18030, ISO 8859-1, etc.

Additionally, when detecting the encoding used in the request from the medical device, the detector 103 may store the terminal identity 1022 of the medical device in the memory 102 in a manner that associates the identity with the detected encoding. For example, the terminal identity 1022 includes, but is not limited to, a device ID and a network address such as an IP address and a MAC address.

Once the detector 103 has detected the encoding of the characters included in the request from the medical device, the converter 104 of the first management server 10 converts the encoding of the characters to the predetermined encoding being used for encoding characters in the medical records. For example, when a request is received from the user terminal 20 for retrieving a list of medical records for a patient named “ [symbol] ” (Wang) encoded by GB18030 encoding, the converter 104 converts the encoding of the patient name to UTF-8 encoding for the subsequent database access query in the first management server 10.

Additionally, when the search result is retrieved, the converter 104 converts the encoding of the characters included in the results to the original encoding used in the request from the medical device. The converter 104 may recognize the original encoding of the request by referring to the terminal identity 1022 stored in the memory 102. As discussed above, in one or more embodiments of the invention, the characters included in the medical database 1021 are encoded in a predetermined encoding such as UTF-8. In that case, the converter 104 converts the characters included in the search results back to the original encoding, i.e., GB18030 encoding. The converted characters may be used to respond to the request from the other medical device.

The database manager 105 of the first management server 10 handles a query to the medical database 1021 and generates the search result. In one or more embodiments of the invention, the database manager 105 searches the medical records in the medical database 1021 based on the converted characters of a request from another medical device. For example, in a case where a request is received from the user terminal 20 for retrieving a list of medical records for a patient name “ [symbol] ” (Wang) encoded in GB18030, the database manager 105 searches the medical database 1021 to list the medical records where the “patient name” attribute matches the patient name “ [symbol] ” (Wang), which has been converted to UTF-8. The result is transmitted to the user terminal 20 after being converted back to the original encoding, i.e., GB18030, via the transceiver 101.

The user terminal 20 of the medical system 1 in accordance with one or more embodiments of the invention includes a transceiver 201, a UI handler 202, and a request generator 203.

The transceiver 201 of the user terminal 20 communicates with the first management server 10. In one or more embodiments of the invention, the transceiver 201 transmits a request for retrieving a list of medical records stored in the first management server 10 and a request for retrieving a medical image, a medical image order, a medical image interpretation report, and a medical examination report, in the form of a DICOM file. The transceiver 201 receives a response (e.g., a list of medical records, a medical image, a medical image order, a medical image interpretation report, and a medical examination report) from the first management server 10 in the form of the DICOM file.

The UI handler 202 of the user terminal 20 handles input from and output to a user interface, such as a graphical user interface shown in FIGS. 6-8. For example, as shown in FIG. 6, the UI handler 202 enables a user of the user terminal 20 to input search criteria including: a study ID, a patient ID, patient's sex, a modality, a study date, a patient name, and a date of birth, and view the search result in the form of a list of the medical records. In the example of FIGS. 6 and 7, only the patient name “ [symbol] ” (Wang) is specified as a sole criterion, and the list indicating three medical images taken for this patient is listed. In this example, the patient name is input in GB18030 encoding, which is a default encoding of the operating system of the user terminal 20.

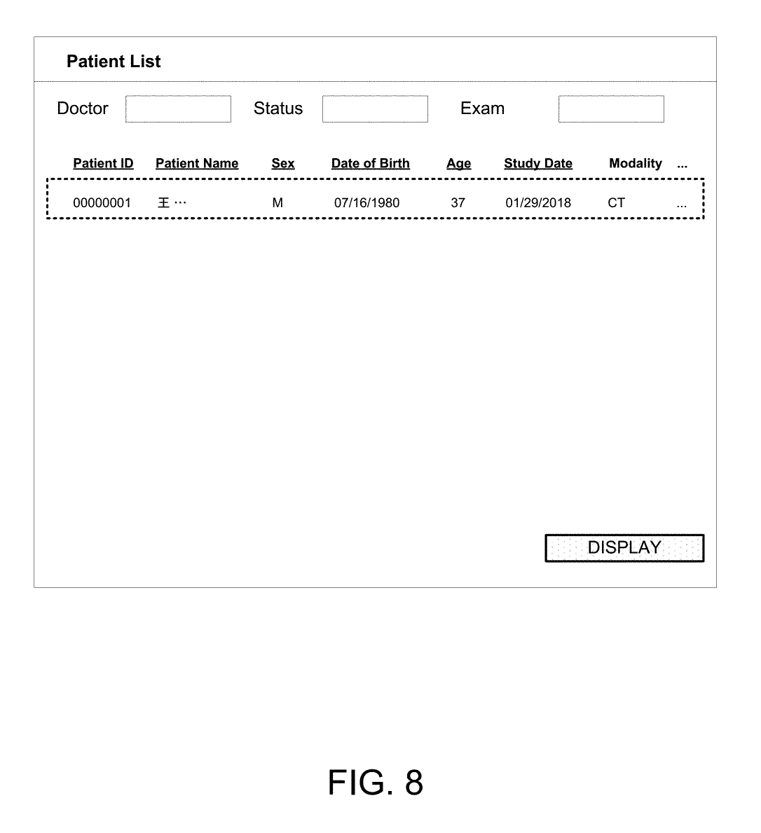

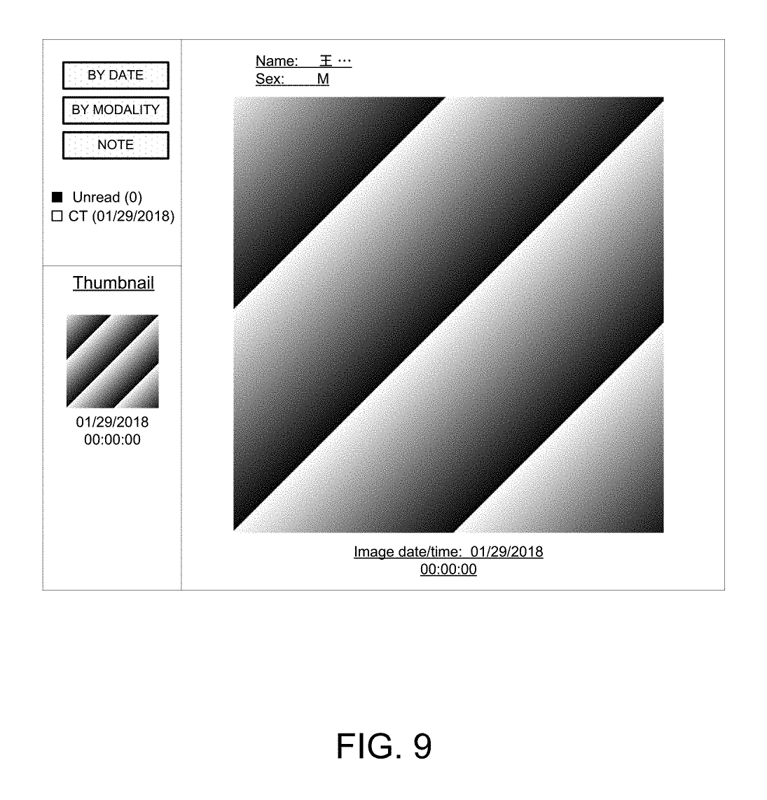

Additionally, as shown in FIG. 7, the UI handler 202 further enables the user to choose and retrieve the actual data of a specific medical record. In the example of FIG. 7, the first medical record for a specific patient is chosen by the user. Once the actual data of the chosen medical record is retrieved, the UI handler 202 may generate another screen as shown in FIG. 8. The UI handler 202 allows the user to select and display one of the chosen records via the screen of FIG. 8, and then allows the user to view the chosen record via the screen of FIG. 9.

The request generator 203 of the user terminal 20 generates a request to be transmitted to the first management server 10 based on user input made via the UI handler 202. In one or more embodiments of the invention, when the search criteria has been entered via the UI handler 202, the request generator 203 generates a request for database search or medical record retrieval based on specified search criteria in the form of a DICOM file.

The imaging device 30 of the medical system 1 in accordance with one or more embodiments of the invention includes a transceiver 301, an image processor 302, and a request generator 303.

The transceiver 301 of the imaging device 30 communicates with the first management server 10. In one or more embodiments of the invention, the transceiver 301 transmits a request for retrieving medical records including medical image orders in the form of a DICOM file.

The image processor 302 of the imaging device 30 takes medical images of a patient and generates digital data encoded with a common image format such as JPEG.

The request generator 303 of the imaging device 30 generates a request for retrieving medical records in response to the operation of an imaging device user. In one or more embodiments of the invention, the request generator 303 generates a request for retrieving a medical image order in the form of a DICOM file.

While FIG. 2 shows one particular configuration of components for illustration purposes, other configurations may be used without departing from the scope of the invention. For example, various components may be combined to form a single component. As another example, the functionality performed by a single component may be performed by two or more components.

Each of the components 102-105 may be located on the same first management server 10 or may be located on different computing devices connected by the network 50 having wired and/or wireless segments. Further, one or more of the components 101-105, 201-203, and 301-303 may be implemented by one or more processors or other dedicated hardware.

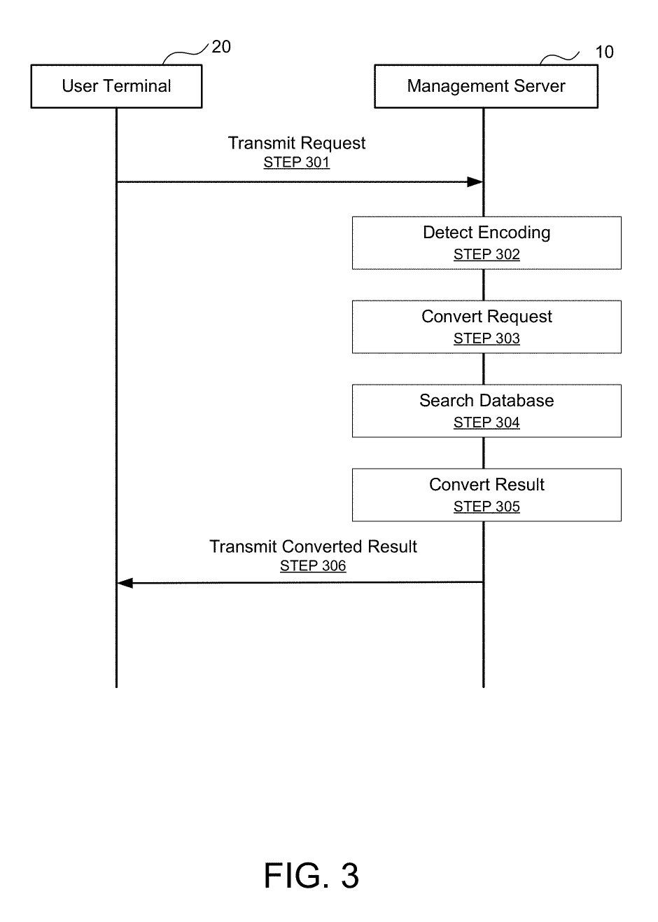

FIG. 3 shows a flowchart in accordance with one or more embodiments of the invention. The process depicted in FIG. 3 may be used for the user terminal 20 to transmit a request for retrieving a list of medical records and to receive the result from the first management server 10. One or more of the steps in FIG. 3 may be performed by the components of the medical system 1, discussed above with reference to FIG. 2. In one or more embodiments of the invention, one or more of the steps shown in FIG. 3 may be omitted, repeated, and/or performed in a different order than the order shown in FIG. 3. Accordingly, the scope of the invention is not limited to the specific arrangement of steps shown in FIG. 3. Although only an example of retrieving the list of medical records in the user terminal 20 is shown, the imaging device 30 or the second management server 40 may request a similar search according to the steps of FIG. 3.

Initially, in response to user's query via the UI shown in FIG. 6, the request generator 203 of the user terminal 20 generates a request for retrieving a list of medical records in the form of a DICOM file, and the transceiver 201 transmits the generated DICOM file to the first management server 10 via the network 50 (S301). In this example, the request generator 203 generates a DICOM file including a request command (e.g., C-Find) for medical content search with keyword characters “ [symbol] ” (Wang), which have been encoded in GB18030 encoding.

When the transceiver 101 of the first management server 10 receives the request (i.e., the DICOM file) from the user terminal 20, the detector 103 parses the DICOM file and detects the encoding of the characters specified as search criteria (S302). In the example of FIG. 6, the detector 103 searches the content of the DICOM file surrounded by tags, and detects the encoding of the keyword characters by locating the predetermined characters “GB18030.”

Once the detector 103 has detected the encoding of the characters included in the request, the converter 104 converts these characters to a predetermined encoding used in the medical database 1021 (S303). In the example of FIG. 6, the converter 104 converts the encoding of the patient name “ [symbol] ” (Wang) to UTF-8 encoding.

Subsequently, the database manager 105 searches the medical records of the medical database 1021 based on the request from the user terminal 20 by using the characters converted by the converter 104 (S304). In the example of FIG. 6, the database manager 105 searches the medical database 1021 to find medical records where the “patient name” attribute matches the patient name “ [symbol] ” (Wang).

When the search is done and one or more medical records that meet the search criteria are found, the converter 104 converts the encoding of the characters included in each of the records to the original encoding used in the request from the user terminal 20 (S305). In the example of FIG. 6, the converter 104 converts the characters included in the search result back to GB18030 encoding.

Finally, the transceiver 101 of the first management server 10 transmits a response including the search result where the characters included have been converted to the encoding used in the original request (S306). In this example, when the transceiver 201 of the user terminal 20 receives the response, the UI handler 202 may display the search result in the form of a list of medical records, as shown in FIG. 7.

According to this configuration, the first management server 10 in accordance with one or more embodiments of the invention allows an existing medical device that operates in a different system language to be integrated into the medical system without modifying existing software.

In one or more embodiments of the invention, at S305, the converter 104 may convert the characters of the search result to another encoding that is different from the original encoding of the request (i.e., the input characters in the user terminal 20) and the encoding of the medical database (i.e., the encoding of characters in the medical records). In this case, the memory 102 stores the terminal identity 1022 associated with the other encoding in advance, and the converter 104 converts the search result based on this information regardless of the detected encoding at S302. For example, the other encoding is preconfigured by a user of the user terminal 20. The other encoding may be an encoding that the user of the user terminal 20 prefers when viewing the search result.

In one or more embodiments of the invention, the characters contained in the request (i.e., DICOM file) from the user terminal 20 may be encoded in two or more encodings. For example, the detector 103 may parse characters surrounded by tags of the DICOM file to find two or more sets of the predetermined characters each indicating the encoding ISO 8859-1 and ISO 8859-2 or JIS X201 and JIS X208. In this case, the converter 104 may convert the characters included in the request encoded with two or more encodings to the predetermined encoding (e.g., UTF-8), and then convert the search result back to each encoding. According to this configuration, the first management server 10 allows the user terminal 20 to request medial content search with search criteria or keywords encoded in two or more encodings.

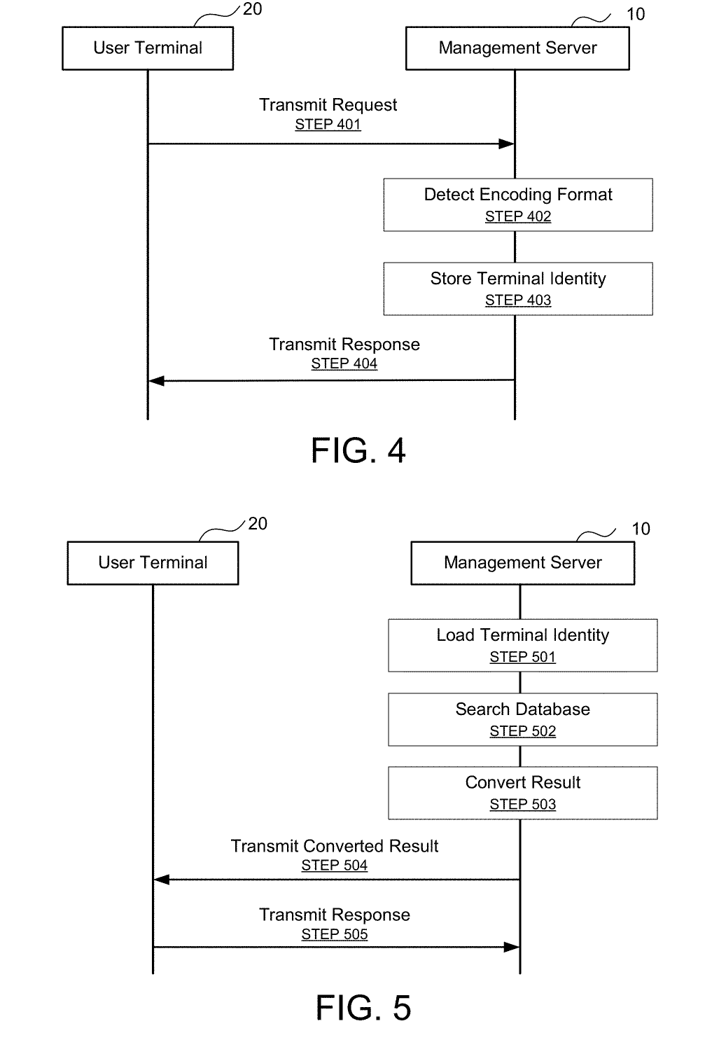

FIGS. 4 and 5 each show a flowchart in accordance with one or more embodiments of the invention. The process depicted in FIGS. 4 and 5 may be used for the user terminal 20 to transmit a request for retrieving one or more medical records including medical images from the first management server 10. One or more of the steps in FIGS. 4 and 5 may be performed by the components of the medical system 1, discussed above with reference to FIG. 2. In one or more embodiments of the invention, one or more of the steps may be omitted, repeated, and/or performed in a different order than the order shown in FIGS. 4 and 5. Accordingly, the scope of the invention is not limited to the specific arrangement of steps shown in FIGS. 4 and 5. Although only an example of retrieving medical images in the user terminal 20 is shown, the other medical devices including the imaging device 30 and the second management server 40 may transmit the similar request for retrieving medical images. Moreover, the user terminal 20 and the other medical devices may transmit a request for retrieving medical image orders, medical image interpretation reports, and medical examination reports according to the steps of FIGS. 4 and 5.

In response to user input via the UI shown in FIG. 7, i.e., the user's selection in the list of medical records, the request generator 203 of the user terminal 20 generates a request for retrieving the selected medical record, and the transceiver 201 transmits the request to the first management server 10 (S401). For example, the request may be formed according to the DICOM file format, including a command for retrieving medical images (e.g., C-MOVE) and characters that identify the medical image.

When the transceiver 101 of the first management server 10 receives the request from the user terminal 20, the detector 103 parses the DICOM file and detects the encoding of the characters (S402). In the example of FIG. 7, the detector 103 searches the content of the DICOM file and detects the encoding of the contained characters by locating the predetermined characters “GB18030.”

Subsequently, the detector 103 stores the terminal identity 1022 of the user terminal 20 in the memory 102 in a manner that associates the identity with the detected encoding (S403). In the example of FIG. 7, the detector 103 may store the encoding “GB18030” associated with the IP address of the user terminal 20.

Once the detector 103 has detected the encoding of the characters in the request and stored its identity in the memory 102, the transceiver 101 transmits an acknowledgement as a response to the user terminal 20 (S404). The acknowledgement itself need not be encoded in the same encoding as the one used in the request from the user terminal 20, because the acknowledgement simply indicates a return code without transmitting any medical record stored in the first management server 10. The logical network session between the user terminal 20 and the first management server 10 ends after the steps shown in FIG. 4.

After the acknowledgement has been transmitted to the user terminal 20, the converter 104 of the first management server 10 loads the terminal identity 1022 of the user terminal 20 (e.g., IP address) and its encoding (S501). Next, the database manager 105 searches the medical database 1021 and identifies the medical record requested by the user terminal 20, based on the characters (i.e., keywords) converted by the converter 104 (S502). Finally, the converter 104 converts the encoding of characters included in the requested medical record (e.g., attributes of a requested medical image) to the original encoding used by the user terminal 20 (S503), and the transceiver 101 transmits to the user terminal 20 the medical record in the form of the DICOM file (S504). The DICOM file may also contain a command “C-STORE” to transmit the medical image to the user terminal 20. The user terminal 20 that has received the medical record transmits an acknowledgement as a response to the first management server 10 (S505). The acknowledgement itself need not be encoded in the same encoding as the one used in the request from the user terminal 20. For example, the acknowledgement may be encoded in the predetermined encoding used for the medical records. FIG. 8 shows a screen that enables the user to choose the retrieved medical image on the display of the user terminal 20, and FIG. 9 shows a screen for the user to view the chosen medical image on the display.

According to this configuration, the first management server 10 may respond to a request for retrieving a medical image and its attributes from the user terminal 20 that operates in a different system language. Additionally, because the first management server 10 maintains the identity of the requesting user terminal 20 associating with the encoding, even after the network session is terminated, the first management server 10 may correctly convert the encoding of the attributes of the medical image to the one used by the user terminal 20.

In one or more embodiments of the invention, at S503, the converter 104 may convert the characters of the search result to another encoding that is different from the original encoding of the request (i.e., the input characters in the user terminal 20) and the encoding of the medical database (i.e., the encoding of characters in the medical records). In this case, the memory 102 stores the terminal identity 1022 associated with the other encoding in advance, and the converter 104 converts the search result based on this information regardless of the detected encoding at S302. For example, the other encoding is preconfigured by a user of the user terminal 20. The other encoding may be an encoding that the user of the user terminal 20 prefers when viewing the search result.

In one or more embodiments of the invention, the characters contained in the request (i.e., DICOM file) from the user terminal 20 may be encoded in two or more encodings. For example, the detector 103 may parse characters surrounded by tags of the DICOM file to find two or more sets of the predetermined characters indicating the encoding, such as GB18030, ISO 8859-1, etc. In this case, the converter 104 may convert the characters included in the request to the predetermined encoding (e.g., UTF-8), and then convert the search result back to each encoding. According to this configuration, the first management server 10 allows the user terminal 20 to request medial content retrieval with search criteria or keywords encoded in two or more encodings.

Embodiments of the invention may be implemented on virtually any type of management server and user terminal, regardless of the platform being used. For example, the first management server 10 may be one or more desktop computers, servers, blades in a server chassis, or any other type of computing device or devices that includes at least the minimum processing power, memory, and input and output devices to perform one or more embodiments of the invention. Additionally, the user terminal 20 may be one or more mobile devices (e.g., laptop computer, smartphone, personal digital assistant, tablet, or other mobile device), desktop computers, servers, blades in a server chassis, or any other type of computing device or devices that includes at least the minimum processing power, memory, and input and output devices to perform one or more embodiments of the invention.

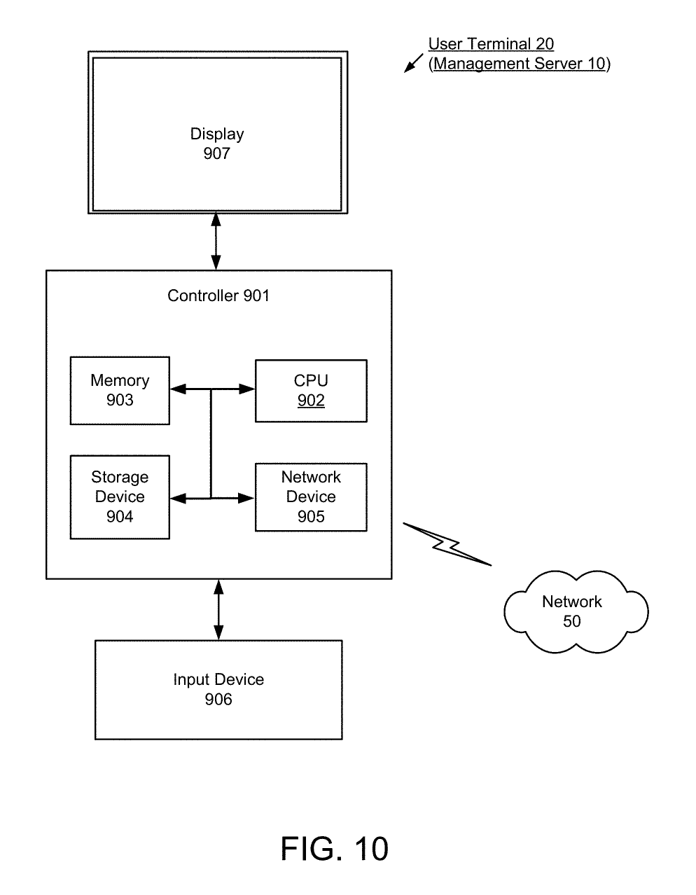

For example, as shown in FIG. 10, the first management server 10 and the user terminal 20 may include one or more CPUs 902, associated memory 903 (e.g., random access memory (RAM), cache memory, flash memory, etc.), one or more storage devices 904 (e.g., a hard disk, a solid state drive, an optical drive such as a compact disk (CD) drive or digital versatile disk (DVD) drive, a flash memory drive, etc.), a network device 905 (e.g., a network interface card, a wireless LAN module, a wide area network module, a Bluetooth module, a ZigBee module, an infra-red communication module, etc.), and numerous other elements and functionalities.

The CPU 902 may be an integrated circuit for processing instructions. For example, the computer processor may be one or more cores or micro-cores of a processor. The CPU 902 may have one or more caches which are faster memories than the (main) memory 903. The first management server 10 and the user terminal 20 may also include one or more input devices 906, such as a keyboard and a mouse or any other type of input device. Further, the first management server 10 and the user terminal 20 may include a display 907. The first management server 10 and the user terminal 20 may also include a network device 905 connected to the network 50 (e.g., a local area network (LAN), a wide area network (WAN) such as the Internet, mobile network, or any other type of network) via a network interface connection (not shown). The input and output devices may be locally or remotely (e.g., via the network 50) connected to the CPU 902, memory 903, storage device 904, and network device 905. Many different types of computing systems exist, and the aforementioned input and output devices may take other forms.

Software instructions in the form of computer readable program code to perform embodiments of the invention may be stored, in whole or in part, temporarily or permanently, on a non-transitory computer readable medium such as a CD, DVD, Blu-ray Disc, storage device, a diskette, a tape, flash memory, physical memory, or any other computer readable storage medium. Specifically, the software instructions may correspond to computer readable program code that, when executed by a processor, is configured to perform embodiments of the invention.

Further, one or more elements of the aforementioned first management server 10 may be located at a remote location and connected to the other elements over a network 50. Further, one or more embodiments of the invention may be implemented on a distributed system having a plurality of nodes, where each portion of the invention may be located on a different node within the distributed system. In one embodiment of the invention, the node corresponds to a distinct computing device. Alternatively, the node may correspond to a computer processor with associated physical memory. The node may alternatively correspond to a computer processor or micro-core of a computer processor with shared memory and/or resources.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A management server that communicates with a plurality of medical devices, comprising:
a memory that stores a plurality of medical records each comprising characters encoded in a first encoding; and
a processor that:
receives a request from one of the plurality of medical devices;
detects a second encoding used to encode characters included in the request;
converts the characters included in the request to the first encoding;
searches the plurality of medical records with the converted characters;
converts a search result of the plurality of medical records to the second encoding; and
transmits the search result to the medical device.

2. The management server of claim 1, wherein
the request is formed according to a predetermined data format for medical images, and
the characters specify an attribute of at least one of the medical images.

3. The management server of claim 2, wherein
the search result comprises either a list of the medical images or one or more medical images specified by the characters.

4. The management server of claim 1, wherein
the medical device is one of: a medical imaging device, a medical user terminal, and a second management server that communicates with the plurality of medical devices used in a plurality of medical facilities.

5. The management server of claim 1, wherein
the second encoding is a default encoding used by the medical device.

6. The management server of claim 1, wherein
when the request is made for retrieving a medical image, the processor transmits a first response to the request without using the second encoding prior to searching the plurality of medical records, and
after receiving the medical image, the one of the medical devices transmits a second response to the search result without using the second encoding.

7. The management server of claim 6, wherein
a first network session is established for the medical device to transmit the request and receive the first response; and
a second network session is established for the management server to transmit the search result and receive the second response.

8. The management server of claim 7, wherein
the processor stores in the memory an identity of the medical device associated with the detected second encoding.

9. The management server of claim 1, wherein
when the processor detects a third encoding either preconfigured or used by a user of the one of the plurality of the medical devices, the processor converts the search result to the third encoding and transmits the search result to the medical device.

10. The management server of claim 1, wherein
the plurality of medical records is one of: medical images, medical image orders, medical image interpretation reports, and medical examination reports.

11. A method for a management server that communicates with a plurality of medical devices, comprising:
storing a plurality of medical records each comprising characters encoded in a first encoding;
receiving a request from one of the plurality of medical devices;
detecting a second encoding used to encode characters included in the request;
converting the characters included in the request to the first encoding;
searching the plurality of medical records with the converted characters;
converting a search result of the plurality of medical records to the second encoding; and
transmitting the search result to the medical device.

12. The method of claim 11, wherein
the request is formed according to a predetermined data format for medical images, and
the characters specify an attribute of at least one of the medical images.

13. The method of claim 12, wherein
the search result comprises either a list of the medical images or one or more medical images specified by the characters.

14. The method of claim 11, wherein
the medical device is one of: a medical imaging device, a medical user terminal, and a second management server that communicates with the plurality of medical devices used in a plurality of medical facilities.

15. The method of claim 11, wherein
the second encoding is a default encoding used by the medical device.

16. The method of claim 11, further comprising
when the request is made for retrieving a medical image, transmitting a first response to the request without using the second encoding prior to searching the plurality of medical records,
wherein, after receiving the medical image, the one of the medical devices transmits a second response to the search result without using the second encoding.

17. The method of claim 16, wherein
a first network session is established for the medical device to transmit the request and receive the first response; and
a second network session is established for the management server to transmit the search result and receive the second response.

18. The method of claim 17, further comprising
storing an identity of the medical device associated with the detected second encoding.

19. The method of claim 11, further comprising
when a third encoding either preconfigured or used by a user of the one of the plurality of the medical devices is detected, converting the search result to the third encoding and transmitting the search result to the medical device.

20. A non-transitory computer readable medium (CRM) storing instructions for a management server that communicates with a plurality of medical devices, the instructions comprising functionality for:
storing a plurality of medical records each comprising characters encoded in a first encoding;
receiving a request from one of the plurality of medical devices;
detecting a second encoding used to encode characters included in the request;
converting the characters included in the request to the first encoding;
searching the plurality of medical records with the converted characters;
converting a search result of the plurality of medical records to the second encoding; and
transmitting the search result to the medical device.
